(19) United States
(12) Patent Application Publication
(10) Pub. No.: US 2017/0165358 A1
(43) Pub. Date: Jun. 15, 2017

(54) METHODS FOR ENHANCING THE IMMUNOSTIMULATION POTENCY OF ALUMINUM SALT-ABSORBED VACCINES

(71) Applicant: The Government of the United States of America as Reprisented by Secretary of the Army, Fort Detrick, MD (US)

(72) Inventors: Carl R. ALVING, Bethesda, MD (US); Jerome H. KIM, Bethesda, MD (US); Mangala RAO, Silver Spring, MD (US)

(21) Appl. No.: 15/127,076

(22) PCT Filed: Jul. 9, 2014
(86) PCT No.: PCT/US14/45940

§ 371 Date: Sep. 19, 2016

Related U.S. Application Data
(60) Provisional application No. 61/969,905, filed on Mar. 25, 2014.

(51) Int. Cl. A61K 39/39; C12N 7/00; A61K 39/145

(52) U.S. Cl. CPC A61K 39/39; A61K 39/145; C12N 7/00; A61K 2039/55572

(57) ABSTRACT

Provided herein are (1) a method of mixing an aluminum salt-adsorbed immunogen with a monophosphoryl lipid A (MPLA)-containing liposome (L(MPLA)), and (2) the resulting immunogenic composition. The resulting immunogenic composition has an enhanced immunostimulation potency compared with either a composition comprising the uncapsulated immunogen mixed with the L(MPLA) or the aluminum salt-adsorbed immunogen alone.

### CROSS REFERENCE TO RELATED APPLICATIONS

This application claims benefit to U.S. Provisional Application No. 61/969,905 filed Mar. 25, 2014.

### U.S. GOVERNMENT RIGHT

This invention was made with government support. The government has certain rights in the invention.

### FIELD

Described herein is a method of enhancing the immunostimulation potency of an aluminum salt-adsorbed immunogen by mixing the aluminum salt-adsorbed immunogen with a liposome comprising monophosphoryl lipid A (MPLA), and the resulting composition thereof.

### BACKGROUND

Optimal beneficial effects of many modern vaccines require the use of vaccine adjuvants that enhance the immune response while maintaining systemic safety and minimal side reactions after injection. The most common form of clinically approved adjuvants is aluminum salts, which were first tested almost ninety years ago. Aluminum salts are currently used in many vaccines, for example, the vaccines against cervical cancer (HPV), hepatitis, polio, tetanus, diphtheria, and seasonal flu. See, e.g., Baylor et al., 2002, Vaccine 20: S18-S23; see also Kristensen, 2012 Summary of Stability data for licensed vaccines, on the Internet at hypertext transfer protocol://www.path.org/publications/files/TS_vaccinestability_table.pdf. Although their precise mechanisms of action remain to be fully understood, these adjuvants have been widely used for many decades in licensed human vaccines. They have a longer record of safety and have been administered to humans in billions of doses. Id.

Nevertheless, the adjuvant effect of aluminum salts varies, e.g., they range from effective to poorly effective or even non-effective. See, e.g., Aprile et al., 1966, Can. J. Public Health 57: 343-60.

### SUMMARY

Accordingly, there remains a need in the field to develop more potent vaccine formulations. For this, new adjuvants may need to be identified and characterized. Alternatively, such a goal may be achieved by enhancing the immunostimulation potency of the pre-existing aluminum salts-adsorbed vaccines. Provided herein is a method of enhancing immunostimulation potency of an aluminum salt-adsorbed immunogen by mixing a monophosphoryl lipid A (MPLA)-containing liposome (L(MPLA)) composition with the aluminum salt-adsorbed immunogen to obtain a composition having enhanced immunostimulation potency. Also described are compositions produced by such methods. For example, provided herein is a HIV vaccine composition comprising aluminum hydroxide gel-adsorbed gp120 protein mixed with L(MPLA), which displays an enhanced immuneresponse, e.g., increased antibody production in immunized subjects.

Accordingly, a method of preparing an immunogenic composition is provided, comprising mixing an aluminum salt-adsorbed immunogen with a monophosphoryl lipid A (MPLA)-containing liposome (L(MPLA)) to obtain the immunogenic composition in a liquid phase, wherein the aluminum salt-adsorbed immunogen comprising an immunogen absorbed by an aluminum salt. The method may further comprise incubating the aluminum salt-adsorbed immunogen and L(MPLA), upon mixing, at a temperature in the range of about 4° C. to about 37° C. for about 30 minutes to about 24 hours, or preferably about 1 hour to about 12 hours.

The method may result in the immunogenic composition having an enhanced immunostimulation potency compared with the aluminum salt-adsorbed immunogen alone. Additionally or alternatively, the method may result in the immunogenic composition has an enhanced immunostimulation potency compared with the encapsulated immunogen mixed with L(MPLA).

In one aspect, the L(MPLA) may be lyophilized. The L(MPLA) may comprise about 50 mM to about 150 mM phospholipids, and the dry weight ratio between the aluminum and the MPLA within the immunogenic composition may be in the range of about 1:110 to about 85:3. The dry weight ratio between the aluminum and the immunogen within the aluminum salt-adsorbed immunogen may be in the range of about 1:30 to about 85:1.

In another aspect, the aluminum salt may be aluminum phosphate, aluminum hydroxide, aluminum potassium sulfate, or any combination thereof. The aluminum salt-adsorbed immunogen may be an aluminum salt-adsorbed vaccine for Haemophilus influenza type b, hepatitis A, hepatitis B, human papillomavirus, pandemic influenza, Japanese encephalitis, meningococcus, pneumococcus, rabies, tetanus toxoid, diphtheria, tetanus, pertussis, polio, Lyme disease, anthrax, typhoid, or combinations thereof.

In a further aspect, the aluminum salt-adsorbed immunogen may comprise aluminum salt-adsorbed HIV-1 protein gp120. Preferably, the aluminum salt in the aluminum salt-adsorbed HIV-1 protein gp120 is aluminum hydroxide.

Also provided is the immunogenic composition prepared by mixing an aluminum salt-adsorbed immunogen with a monophosphoryl lipid A (MPLA)-containing liposome (L(MPLA)). The immunogenic composition may further comprise a physiologically acceptable vehicle. The immunogenic composition may comprise an aluminum hydroxide-adsorbed HIV-1 protein gp120 as the aluminum salt-adsorbed immunogen, and a single dose of the immunogenic composition may further comprise: (1) about 10 μg to about 600 μg of gp120 protein; (2) about 20 μg to about 850 μg of aluminum; and (3) about 30 μg to about 2.2 mg of L(MPLA) comprising about 50 mM to about 150 mM phospholipids.

A method of enhancing an immunostimulation potency of an aluminum salt-adsorbed immunogen is also provided, comprising mixing L(MPLA) to the aluminum salt-adsorbed immunogen to obtain an immunogenic composition in a liquid phase, wherein the aluminum salt-adsorbed immunogen comprising an immunogen absorbed by an aluminum salt. The method may further comprise incubating the aluminum salt-adsorbed immunogen and L(MPLA), upon mixing, at a temperature in the range of about 4° C. to about 37° C. for about 30 minutes to about 24 hours, or preferably about 1 hour to about 12 hours.

In one aspect, the L(MPLA) may be lyophilized. The L(MPLA) may comprise about 50 mM to about 150 mM phospholipids, and the dry weight ratio between the aluminum and the MPLA within the immunogenic composition may be in the range of about 1:110 to about 85:3. The dry weight ratio between the aluminum and the immunogen within the aluminum salt-adsorbed immunogen may be in the range of about 1:30 to about 85:1.

In another aspect, the aluminum salt may be aluminum phosphate, aluminum hydroxide, aluminum potassium sulfate, or any combination thereof. The aluminum salt-adsorbed immunogen may be an aluminum salt-adsorbed vaccine for Haemophilus influenza type b, hepatitis A, hepatitis B, human papillomavirus, pandemic influenza, Japanese encephalitis, meningococcus, pneumococcus, rabies, tetanus toxoid, diphtheria, tetanus, pertussis, polio, Lyme disease, anthrax, typhoid, or combinations thereof.

In a further aspect, the aluminum salt-adsorbed immunogen may comprise aluminum salt-adsorbed HIV-1 protein gp120. Preferably, the aluminum salt in the aluminum salt-adsorbed HIV-1 protein gp120 is aluminum hydroxide.

Also provided is a use of a L(MPLA) composition to enhance immunostimulation potency of an aluminum salt-adsorbed immunogen.

### BRIEF DESCRIPTION OF THE DRAWING

The accompanying drawing is incorporated into the specification and provide non-limiting illustration of various embodiments.

FIG. 1 illustrates the resulting complex produced by mixing AIDSVAX® (an experimental HIV vaccine comprising HIV-1 gp120) with L(MPLA) as described in Example 1.

### DETAILED DESCRIPTION

### 1. Definitions

An “immunogen” is an agent capable of inducing humoral and/or cell-mediated immune response. The immunogen as described herein can be an antigen or an inactivated pathogen. An immunogenic composition as described herein can be, for example, a vaccine formulation.

“Aluminum salts” used for adjuvants can comprise aluminum phosphate, aluminum hydroxide, aluminum potassium sulfate (alum), or any combination thereof. In the vaccine field, all aluminum salt adjuvants, regardless of exact chemical composition, are commonly referred to informally as “alum.”

“Liposomes” as used herein refer to closed bilayer membranes containing an entrapped aqueous volume. Liposomes may also be unilamellar vesicles possessing a single membrane bilayer or multilamellar vesicles with multiple membrane bilayers, each separated from the next by an aqueous layer. The structure of the resulting membrane bilayer is such that the hydrophobic (non-polar) tails of the lipid are oriented toward the center of the bilayer while the hydrophilic (polar) heads orient towards the aqueous phase. Liposomes, as they are ordinarily used, consist of smectic mesophases, and can consist of either phospholipid or nonphospholipid smectic mesophases. Smectic mesophase is most accurately described by Small, HANDBOOK OF LIPID RESEARCH, Vol. 4, Plenum, N Y, 1986, pp. 49-50. According to Small, “[w]hen a given molecule is heated, instead of melting directly into an isotropic liquid, it may instead pass through intermediate states called mesophases or liquid crystals, characterized by residual order in some directions but by lack of order in others . . . . In general, the molecules of liquid crystals are somewhat longer than they are wide and have a polar or aromatic part somewhere along the length of the molecule. The molecular shape and the polar-polar, or aromatic, interaction permit the molecules to align in partially ordered arrays . . . . These structures characteristically occur in molecules that possess a polar group at one end. Liquid crystals with long-range order in the direction of the long axis of the molecule are called smectic, layered, or lamellar liquid crystals . . . . In the smectic states the molecules may be in single or double layers, normal or tilted to the plane of the layer, and with frozen or melted aliphatic chains.”

Lipid A is a set of complex, heavily acylated and amidated diglucosamine diphosphate molecules and is the lipid moiety common to all lipopolysaccharides (LPS; also known as endotoxin) from Gram-negative bacteria. LPS covers virtually the entire outer surface of all Gram-negative bacteria, and lipid A anchors the LPS into the outer lipid surface of the bacterium. The O-polysaccharide portion of LPS in wild-type smooth bacteria is linked to a relatively conserved core oligosaccharide that is expressed in rough mutants, and this in turn is linked to lipid A through highly conserved 2-keto-3-deoxyoctanoic acid sugars that are unique chemical structures required for bacterial viability and found only in LPS. See, e.g., Alving et al., 2012, Expert Rev. Vaccines 11: 733-44. “Monophosphoryl lipid A” is a lipid A congener in which the glucosamine-1-phosphate group on the polar head group has been removed. Numerous congeners of MPLA also exist.

A “physiologically acceptable vehicle” as used herein refers to a vehicle that is suitable for in vivo administration (e.g., oral, transdermal or parenteral administration) or in vitro use, i.e., cell culture. Exemplary physiologically acceptable vehicles can be those physiologically acceptable constituents of liposomes as disclosed in U.S. Pat. Nos. 4,186,183 and 4,302,459.

The term “about” as used herein refers to ±15% of the referenced value.

Unless defined otherwise, all technical and scientific terms used herein have the same meaning as commonly understood by one of ordinary skill in the art. It must be noted that as used herein, the singular forms “a”, “and”, and “the” include plural referents unless the context clearly dictates otherwise. Thus, for example, reference to “an antibody” includes a plurality of such antibodies and reference to “the dosage” includes reference to one or more dosages and equivalents thereof known to those skilled in the art, and so forth.

“Preferred” and “Preferably” as used herein are to be construed for purposes of claim construction in Europe only. The terms should be read out of or omitted from the construction of the sentences and paragraphs in which they appear for purposes of U.S. claim construction.

### 2. Aluminum Salt-Adsorbed Vaccines

Aluminum salts used for adjuvants can comprise aluminum phosphate, aluminum hydroxide, aluminum potassium sulfate (alum), or any combination thereof. An exemplary list of aluminum salt-adsorbed vaccines is shown below:
DTaP (for Diphtheria, Tetanus, and Pertussis vaccine)
DTP (for Diphtheria, Tetanus, and Pertussis vaccine)
Hib conjugate (Haemophilus influenza type b, Hib)
Pneumo conjugate (pneumococcal vaccine)
DTP-Hib (combination vaccine for Diphtheria and Haemophilus influenza type b)
Hep B-Hib (combination vaccine for Hepatitis B/Haemophilus influenza type B)
Hep B (Hep B stands for hepatitis B)
DT adsorbed (Diphtheria and tetanus toxoids adsorbed)
T, adsorbed (for Tetanus)
Td, adsorbed (Td stands for Tetanus and Diphtheria)
Hep A (for hepatitis A)
Lyme
Anthrax
Rabies
   See Baylor et al., 2002, Vaccine 20: S18-S23. A more expanded list is provided in Kristensen, 2012, Summary of Stability data for licensed vaccines, on the Internet at hypertext transfer protocol://www.path.org/publications/files/TS_vaccine_stability_table.pdf

At least 146 licensed vaccines exist currently against single or multiple pathogens have been currently adjuvanted with an aluminum salt. Exemplary vaccines include, but are not limited to, those for Haemophilus influenza type b, hepatitis A, hepatitis B, human papillomavirus, pandemic influenza, Japanese encephalitis, meningococcus, pneumococcus, rabies, tetanus toxoid, diphtheria, tetanus, pertussis, polio, Lyme disease, anthrax, typhoid, and combinations thereof. Preferably, the aluminum salt-adsorbed vaccine is provided as an aqueous suspension.

The actual amount of the aluminum salt adjuvant in vaccines may vary depending on multiple factors, e.g., the subject (animal versus human, adult versus child) to be immunized and the route of administration. Immunogenic dosages can be determined by those of skill in the art. In the vaccines licensed in the U.S., the amount of aluminum ranges from about 0.125-0.85 mg/dose. See, Baylor et al., 2002, Vaccine 20: S18-S23. For human vaccination, the preferable range of the amount of aluminum may range from about 20 μg to about 850 μg per dose of vaccine. The amount of immunogen, most commonly protein antigen, may be in the range of about 1 μg to about 1 mg per dose of vaccine, or preferably about 10 μg to about 600 μg per dose of vaccine.

Typically, the immune response by the aluminum salt-adsorbed vaccines can be detected by the presence of antibodies that specifically bind to a particular polypeptide. Methods of detecting antibodies are known to those of skill in the art and include such assays as enzyme-linked immunosorbent assay (ELISA), Enzyme-Linked ImmunoSpot (ELISPOT) assays, Western blot assays, and competition assays.

### 3. Monophosphoryl Lipid A (MPLA)-Containing Liposomes (L(MPLA))

Liposomes are closed bilayer membranes containing an entrapped aqueous volume. Liposomes may also be unilamellar vesicles possessing a single membrane bilayer or multilamellar vesicles with multiple membrane bilayers, each separated from the next by an aqueous layer. The structure of the resulting membrane bilayer is such that the hydrophobic (non-polar) tails of the lipid are oriented toward the center of the bilayer while the hydrophilic (polar) heads orient towards the aqueous phase. Suitable hydrophilic polymers for surrounding the liposomes include, without limitation, PEG, polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxethylacrylate, hydroxymethylcellulose, hydroxyethylcellulose, polyethyleneglycol, polyaspartamide and hydrophilic peptide sequences as described in U.S. Pat. Nos. 6,316,024; 6,126,966; 6,056,973; and 6,043,094. Liposomes can be made without hydrophilic polymers. Therefore, liposome formulations may or may not contain hydrophilic polymers.

Liposomes may be comprised of any lipid or lipid combination known in the art. For example, the vesicle-forming lipids may be naturally-occurring or synthetic lipids, including phospholipids, such as phosphatidylcholine, phosphatidylethanolamine, phosphatidic acid, phosphatidylserine, phosphatidylglycerol, phosphatidylinositol, and sphingomyelin as disclosed in U.S. Pat. Nos. 6,056,973 and 5,874,104.

The vesicle-forming lipids may also be glycolipids, cerebrosides, or cationic lipids, such as 1,2-dioleyloxy-3-(trimethylamino)propane (DOTAP); N-[1-(2,3,-ditetradecyloxy)propyl]-N,N-dimethyl-N-hydroxyethylammonium bromide (DMRIE); N-[1[(2,3,-dioleyloxy)propyl]-N,N-dimethyl-N-hydroxy ethylammonium bromide (DORIE); N-[1-(2,3-dioleyloxy)propyl]-N,N,N-trimethylammonium chloride (DOTMA); 3 [N—(N′,N′-dimethylaminoethane) carbamoly] cholesterol (DCChol); or dimethyldioctadecylammonium (DDAB) also as disclosed in U.S. Pat. No. 6,056,973. Cholesterol may also be present in the proper range to impart stability to the liposome vesicle, as disclosed in U.S. Pat. Nos. 5,916,588 and 5,874,104. Additional liposomal technologies are described in U.S. Pat. Nos. 6,759,057; 6,406,713; 6,352,716; 6,316,024; 6,294,191; 6,126,966; 6,056,973; 6,043,094; 5,965,156; 5,916,588; 5,874,104; 5,215,680; and 4,684,479. These described liposomes and lipid-coated microbubbles, and methods for their manufacture. Thus, one skilled in the art, considering both the present disclosure and the disclosures of these other patents could produce a liposome for the purposes of the present embodiments. For the present embodiments, the liposomes preferably contain 50-150 mM phospholipids.

Any of the above exemplary liposomes would include monophosphoryl Lipid A (MPLA), or could be combined with other liposomes and Lipid A (MPLA). MPLA alone can be toxic to humans and animals. However, when present in liposomes, the toxicity is not detected. See, e.g., Alving et al., 2012, Expert Rev. Vaccines 11: 733-744. Exemplary procedures for preparation of the liposomes with MPLA as described herein are taught at least in Alving et al., 2012, Expert Rev. Vaccines 11: 733-744. MPLA serves as a potent adjuvant and serves to raise the immunogenicity of the liposome and peptides, proteins, or haptens associated with the liposome. For the present embodiments, the amount of MPLA preferably may be in the range of about 30 μg to about 2.2 mg per dose of vaccine.

### Examples

The following examples are provided in order to demonstrate and further illustrate certain representative embodiments and aspect of the present disclosure and are not to be construed as limiting the scope of the specification or claims.

### Materials and Methods

### Immunization

AIDSVAX® (VaxGen, South San Francisco, Calif., U.S.) is an experimental HIV vaccine comprising the HIV surface glycoprotein gp120 as described in Adis International Ltd., 2003, Drugs R. D. 4: 249-53. L(MPLA) was prepared as described in Wassef et al., 1994, ImmunoMethods 4: 217-22.

AIDSVAX® B/E comprises a mixture of clades B and E HIV gp120 proteins adsorbed to aluminum hydroxide (GSID, South San Francisco, Calif., U.S.). Varying amounts of AIDSVAX® B/E were added to lyophilized vials of L(MPLA), and the mixture was left at 4° C. for 1 hour or at 4° C. overnight. Each vial was swirled to ensure that there were no clumps of the lyophilized material as observed by visual inspection. Test articles (50 μl/mouse) were injected intramuscularly by needle and syringe into 9 groups of female BALB/c mice (6 mice per group) as shown in Table 1 below:

TABLE 1
Immunization set up

| | AIDSVAX ® B/E | | L(MPLA) | |
|---|---|---|---|---|
| | Amount | Al Amount | Amount | Mixing and Immunizing |
| Group | μg/dose/50 μl | μg/dose/50 μl | μg/dose/50 μl | Procedure |
| 1 | 30 | 30 | 0 | n/a (not applicable) |
| 2 | 30 | 30 | 9.25 | Inject 12 hr after addition |
| | | | | of AIDSVAX ® BE to |
| | | | | lyophilized L(MPLA) |
| | | | | vial |
| 3 | 30 | 30 | 9.25 | Inject 24-26 hr after |
| | | | | addition of AIDSVAX ® |
| | | | | B/E to lyophilized |
| | | | | L(MPLA) vial |
| 4 | 10 | 10 | 0 | n/a (not applicable) |
| 5 | 1 | 1 | 0 | n/a (not applicable) |
| 6 | 0.1 | 0.1 | 0 | n/a (not applicable) |
| 7 | 10 | 10 | 9.25 | Inject 24-26 hr after |
| | | | | addition of AIDSVAX ® |
| | | | | B/E to lyophilized |
| | | | | L(MPLA) vial |
| 8 | 1 | 1 | 9.25 | Inject 24-26 hr after |
| | | | | addition of AIDSVAX ® |
| | | | | B/E to lyophilized |
| | | | | L(MPLA) vial |
| 9 | 0.1 | 0.1 | 9.25 | Inject 24-26 hr after |
| | | | | addition of AIDSVAX ® |
| | | | | B/E to lyophilized |
| | | | | L(MPLA) vial |

The amounts of gp120 proteins and aluminum salt, as expressed in Table 1, refer to the dry weight. The resulting mixture is in a liquid phase, wherein the lyophilized L(MPLA) has been spontaneously hydrated given that the aluminum salt-adsorbed gp120 was provided as an aqueous suspension.

Mice were immunized through the intramuscular route on weeks 0, 3, 6, and bled on weeks 0, 2, 4, 6, 8, and 10. Individual serum samples were tested by ELISA for IgG binding antibodies to A244 gp120 and MN gp120 proteins (proteins present in AIDSVAX® B/E) at the time points indicated.

Detection of Antibody Responses after Vaccination by ELISA

Ninety-six well U-bottom ELISA plates were coated overnight at 4° C. with 100 μl/well of purified A244 or MN proteins provided by Global Solutions for Infectious Diseases (South San Fransico, Cal., U.S.) as described in Karasavvas et al., 2012, AIDS Res. Hum. Retroviruses 28: 1444-57. The protein was removed and each well was blocked with 250 μl of blocking buffer (Phosphate buffered saline (PBS) containing 0.5% casein and 0.5% bovine serum albumin (BSA), pH 7.4) overnight at 4° C. The plates were washed twice with PBS containing 0.1% Tween-20, pH 7.4 (PBS-T), and 100 μl of serum (1:200 dilution) was added to wells in triplicate and then serially diluted two-fold in blocking buffer. The plates were incubated for 2 hours at room temperature and washed four times with PBS-T. The plates were washed and 100 μl of horseradish peroxidase-conjugated sheep anti-mouse IgG (BindingSite, San Diego, Calif., U.S.) diluted 1:1000 in the blocking buffer were added to each well. The plates were incubated for 1 hour at room temperature, washed, and 100 μl of ABTS (2,2′-azino-bis(3-ethylbenzothiazoline-6-sulphonic acid)) substrate (KPL, Gaithersburg, Md., U.S.) were added to each well. The plates were then incubated for 1 hour in the dark at room temperature. The absorbance was read at 405 nm on an ELISA plate reader.

### Example 1—Addition of L(MPLA) to Aluminum Hydroxide-Adsorbed HIV-1 Gp120 (AIDSVAX® B/E) Resulted in Increased Antibody Titers

The adjuvant field has evolved a number of adjuvant candidates, and the most effective of these are administered as adjuvant formulations that include more than one adjuvant or carrier molecule. In an Acquired Immunodeficiency Syndrome (AIDS) Vaccine Evaluation Group Study 015 (AVEG015), seven adjuvants (including aluminum hydroxide, identified as “alum”) were compared for safety and for the ability to induce immune responses in humans against HIV-1 envelope protein gp120. McElrath, 1995, Semin. Cancer Biol. 6: 375-85. It was observed by McElrath during the AVEG015 study that alum-adsorbed liposomes containing encapsulated gp120 and monophosphoryl lipid A outperformed alum-adsorbed gp120 and performed as well, or better than, each of the other adjuvants for inducing an immune response to gp120, and that these same alum-adsorbed liposomes exhibited low levels of local and systemic toxicity equivalent to the low levels of alum-adsorbed gp120 alone.

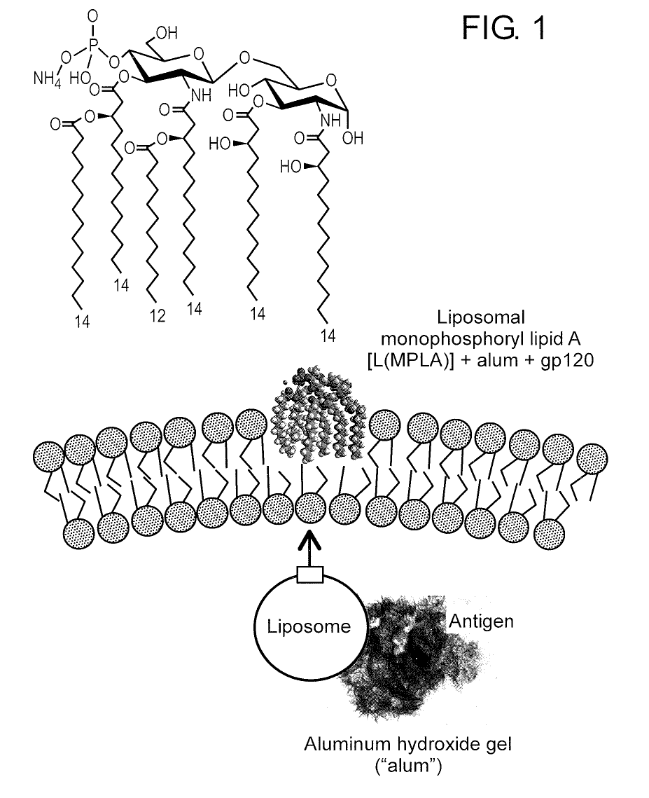

Given the McElrath's results, the following experiments were conducted to evaluate the effects of directly mixing alum-adsorbed gp120 with liposomes containing MPLA, i.e., gp120 is not encapsulated within the liposomes (FIG. 1), different from what is described in McElrath. For this, female BALB/c mice of 6-8 weeks were immunized as described in the Materials and Methods, as illustrated in Table 1. Blood samples were collected for each mouse at weeks 0, 2, 4, 6, 8, and 10. ELISA was performed to determine the titers of antibodies in the sera as described in the Materials and Methods above. The arithmetic mean and the standard error of the mean (SEM) for each group at each time point were calculated, and the data are compiled in Table 2 below:

TABLE 2
Compiled antibody titers

| | 2 weeks post 1^{st} | | 1 week post 2nd | | 3 weeks post 2nd | | 2 weeks post 3rd | | 4 weeks post 3rd | |
|---|---|---|---|---|---|---|---|---|---|---|
| | immunization | | immunization | | immunization | | immunization | | immunization | |
| Group | A244 | MN | A244 | MN | A244 | MN | A244 | MN | A244 | MN |
| 1 | 833 ± | 1,467 ± | 34,133 ± | 115,200 ± | 20,267 ± | 68,267 ± | 78,933 ± | 955,733 ± | 119,467 ± | 750,933 ± |
| | 182 | 382 | 7,867 | 30,826 | 3,473 | 27,782 | 27,733 | 136,533 | 28,558 | 195,486 |
| 2 | 2,267 ± | 4,267 ± | 68,267 ± | 136,533 ± | 59,733 ± | 115,200 ± | 315,733 ± | 887,467 ± | 256,000 ± | 887,467 ± |
| | 434 | 675 | 10,794 | 21,588 | 8,533 | 59,764 | 110,933 | 164,408 | 51,200 | 164,408 |
| 3 | 2,800 ± | 4,533 ± | 59,733 ± | 71,680 ± | 64,000 ± | 59,733 ± | 163,840 ± | 1,058,133 ± | 187,733 ± | 614,400 ± |
| | 820 | 868 | 8,533 | 12,541 | 12,800 | 29,313 | 25,083 | 271,784 | 48,871 | 91,589 |
| 4 | 480 ± | 750 ± | 16,533 ± | 34,880 ± | 12,267 ± | 9,067 ± | 42,667 ± | 290,133 ± | 46,933 ± | 375,467 ± |
| | 136 | 309 | 7,681 | 19,015 | 4,538 | 1,736 | 5,397 | 116,504 | 4,267 | 107,939 |
| 5 | ND | ND | 3,633 ± | 2,467 ± | 3,867 ± | 3,333 ± | 14,933 ± | 104,533 ± | 19,200 ± | 113,067 ± |
| | | | 1,900 | 1,266 | 2,004 | 1,982 | 3,570 | 33,771 | 2,862 | 32,113 |
| 6 | 200 ± | 800 ± | 200 ± | ND | 867 ± | 267 ± | 1,840 ± | 9,067 ± | 4.467 ± | 11,233 ± |
| | 0 | 0 | 0 | | 405 | 123 | 588 | 2,397 | 1,883 | 3,652 |
| 7 | 2,200 ± | 3,067 ± | 59,733 ± | 145,100 ± | 55,467 ± | 98,133 ± | N/A | 324,267 ± | N/A | 324,267 ± |
| | 482 | 784 | 8,533 | 58,282 | 10,275 | 33,972 | | 107,126 | | 55,565 |
| 8 | 467 ± | 1,100 ± | 41,600 ± | 55,467 ± | 10,933 ± | 20,267 ± | N/A | 256,000 ± | N/A | 375,467 ± |
| | 111 | 300 | 14,382 | 10,275 | 3,417 | 3,473 | | 51,200 | | 97,742 |
| 9 | 800 ± | 200 ± | 1,800 ± | 960 ± | 10,680 ± | 17,933 ± | N/A | 27,733 ± | N/A | 38,400 ± |
| | 0 | 0 | 503 | 299 | 10,133 | 16,901 | | 7,692 | | 8,095 |

N/A: not available;
ND: not detectable by ELISA.

According to the results in Table 2, addition of L(MPLA) to AIDSVAX® B/E resulted in a dramatic increase in IgG antibodies specific to A244 and MN. The multi-fold increase varies between 2.7-12-fold increase depending upon the weeks post immunization and the amount of the antigen used during immunization. Immunization of mice with 1 μg of AIDSVAX® B/E containing L(MPLA) induced antibody responses that were equivalent to antibody responses induced after immunization of mice with 10 μg of AIDSVAX® B/E lacking the L(MPLA). Thus, a smaller dose of antigen (dose sparing of antigen) induced similar responses when L(MPLA) was also present. In all cases, immunization of mice with AIDSVAX® B/E containing L(MPLA) showed higher antibody titers to both A244 and MN proteins when compared to AIDSVAX® B/E alone. Additionally, there appeared to be no difference whether the addition of AIDSVAX® B/E to the lyophilized L(MPLA) was carried out for 1 hour or overnight, because the antibody titers appeared similar.

The method of mixing an aluminum salt-adsorbed vaccine, such as anyone of those taught in Baylor et al., 2002 and Kristensen, 2012, with the L(MPLA) described here is believed to enhance the immunostimulation potency of each vaccine composition, not just the vaccine composition exemplified. The methods described herein may enable a greater ease of utilizing liposomal MPLA as an adjuvant for a premade aluminum salt-adsorbed protein vaccine.

The present finding, i.e., the enhanced immunostimulation potency by mixing an aluminum salt-adsorbed vaccine with L(MPLA), is surprising. It was known that the presence of an aluminum salt adjuvant could disrupt liposomes and cause structural changes in the liposomal membrane, ultimately resulting in a reduced immune response. See U.S. Pat. No. 5,820,880. Additionally, the reasons for the disruption of the liposomes by aluminum salts remain unclear. Accordingly, those of skill in the art likely would have been discouraged from mixing any aluminum salt-adsorbed vaccine with L(MPLA).

What is claimed is:

1: A method of preparing an immunogenic composition comprising mixing an aluminum salt-adsorbed immunogen with a monophosphoryl lipid A (MPLA)-containing liposome (L(MPLA)) to obtain the immunogenic composition in a liquid phase, wherein the aluminum salt-adsorbed immunogen comprising an immunogen absorbed by an aluminum salt.

2: The method of claim 1 further comprising incubating the aluminum salt-adsorbed immunogen and L(MPLA), upon mixing, at a temperature in the range of about 4° C. to about 37° C. for about 30 minutes to about 24 hours.

3: The method of claim 1, wherein the L(MPLA) is lyophilized.

4: The method of claim 1, wherein the L(MPLA) comprises about 50 mM to about 150 mM phospholipids, and wherein the dry weight ratio between the aluminum and the MPLA within the immunogenic composition is in the range of about 1:110 to about 85:3.

5: The method of claim 1, wherein the dry weight ratio between the aluminum and the immunogen within the aluminum salt-adsorbed immunogen is in the range of about 1:30 to about 85:1.

6: The method of claim 1, wherein the aluminum salt is aluminum phosphate, aluminum hydroxide, aluminum potassium sulfate, or any combination thereof.

7: The method of claim 1, wherein the aluminum salt-adsorbed immunogen is an aluminum salt-adsorbed vaccine for Haemophilus influenza type b, hepatitis A, hepatitis B, human papillomavirus, pandemic influenza, Japanese encephalitis, meningococcus, pneumococcus, rabies, tetanus toxoid, diphtheria, tetanus, pertussis, polio, Lyme disease, anthrax, typhoid, or combinations thereof.

8: The method of claim 1, wherein the aluminum salt-adsorbed immunogen comprises aluminum salt-adsorbed HIV-1 protein gp120.

9: The method of claim 1, wherein the aluminum salt is aluminum hydroxide.

10: The method of claim 1, wherein the immunogenic composition has an enhanced immunostimulation potency compared with the aluminum salt-adsorbed immunogen alone.

11: The method of claim 1, wherein the immunogenic composition has an enhanced immunostimulation potency compared with the uncapsulated immunogen mixed with L(MPLA).

12: The immunogenic composition prepared by the method of claim 1.

13: The immunogenic composition of claim 12, further comprising a physiologically acceptable vehicle.

14: The immunogenic composition of claim 12, wherein the aluminum salt-adsorbed immunogen is an aluminum hydroxide-adsorbed HIV-1 protein gp120, and wherein a single dose of the immunogenic composition further comprises:
about 10 μg to about 600 μg of gp120 protein;
about 20 μg to about 850 μg of aluminum; and
about 30 μg to about 2.2 mg of L(MPLA) comprising about 50 mM to about 150 mM phospholipids.

15: A method of enhancing an immunostimulation potency of an aluminum salt-adsorbed immunogen comprising mixing L(MPLA) to the aluminum salt-adsorbed immunogen to obtain an immunogenic composition in a liquid phase, wherein the aluminum salt-adsorbed immunogen comprising an immunogen absorbed by an aluminum salt.

16: The method of claim 15 further comprising incubating the aluminum salt-adsorbed immunogen and L(MPLA), upon mixing, at a temperature in the range of about 4° C. to about 37° C. for about 30 minutes to about 24 hours.

17: The method of claim 15, wherein the L(MPLA) is lyophilized.

18: The method of claim 15, wherein the L(MPLA) comprises about 50 mM to about 150 mM phospholipids, and wherein the dry weight ratio between the aluminum and the MPLA within the immunogenic composition is in the range of about 1:110 to about 85:3.

19: The method of claim 15, wherein the dry weight ratio between the aluminum and the immunogen within the aluminum salt-adsorbed immunogen is in the range of about 1:30 to about 85:1.

20: The method of claim 15, wherein the aluminum salt is aluminum phosphate, aluminum hydroxide, aluminum potassium sulfate, or any combination thereof.

21: The method of claim 15, wherein the aluminum salt-adsorbed immunogen is an aluminum salt-adsorbed vaccine for Haemophilus influenza type b, hepatitis A, hepatitis B, human papillomavirus, pandemic influenza, Japanese encephalitis, meningococcus, pneumococcus, rabies, tetanus toxoid, diphtheria, tetanus, pertussis, polio, Lyme disease, anthrax, typhoid, or combinations thereof.

22: The method of claim 15, wherein the aluminum salt-adsorbed immunogen comprises aluminum salt-adsorbed HIV-1 protein gp120.

23: The method of claim 15, wherein the aluminum salt is aluminum hydroxide.

24: Use of a L(MPLA) composition to enhance immunostimulation potency of an aluminum salt-adsorbed immunogen.
